(19) United States
(12) Patent Application Publication
(10) Pub. No.: US 2018/0233228 A1
(43) Pub. Date: Aug. 16, 2018

(54) SYSTEMS AND METHODS FOR DATA-DRIVEN MEDICAL DECISION MAKING ASSISTANCE

(71) Applicants: GilAnthony Ungab, San Diego, CA (US); Mathew Rothway, San Francisco, CA (US); Jeremy Martinson, La Mesa, CA (US)

(72) Inventors: GilAnthony Ungab, San Diego, CA (US); Mathew Rothway, San Francisco, CA (US); Jeremy Martinson, La Mesa, CA (US)

(21) Appl. No.: 15/896,479

(22) Filed: Feb. 14, 2018

Related U.S. Application Data
(60) Provisional application No. 62/458,977, filed on Feb. 14, 2017.

(51) Int. Cl. G16H 50/20; G16H 40/67; G06T 7/00; A61B 5/1455; A61B 5/022; A61B 6/03; A61B 5/00

(52) U.S. Cl. CPC G16H 50/20; G16H 40/67; G06T 7/0012; A61B 5/14551; A61B 5/0077; A61B 6/032; A61B 5/7275; G06T 2207/20081; A61B 5/022

(57) ABSTRACT

Systems, methods, and computer-readable media for providing medical recommendations are disclosed. The system includes one or more data capture devices for collecting captured data that indicates a medical condition of a subject. The system also includes one or more processors and one or more non-transitory memory modules communicatively coupled to the one or more processors and the one or more data capture devices. The memory modules store machine-readable instructions that, when executed, cause the one or more processors to receive the captured data and the image data from the one or more data capture devices. The processors are further caused to complete one or more decision-making processes by analyzing the captured data and the image data. The computing device is trained to analyze the captured data indicating the medical condition of the subject and the visual data pertaining to the at least one characteristic of the subject.

### CROSS-REFERENCE To RELATED APPLICATION

The present application claims priority to U.S. Provisional Patent Application Ser. No. 62/458,977, filed Feb. 14, 2017 and entitled “Systems and Methods for Continuously Monitoring a Temperature of An Electrical Supply System,” which is incorporated by reference herein in its entirety.

### TECHNICAL FIELD

The present specification generally relates to systems and methods for assisting medical personnel in making medical decisions and, more specifically, to systems and methods that use an artificial intelligence system to assist medical personnel with making decisions relating to long term care, decisions that are based on promulgated medical guidelines, medical prescription decisions based on a particular insurance formulary, and/or the like.

### BACKGROUND

Currently, when a subject is presented to medical personnel, a diagnosis may be made based on the medical personnel's medical knowledge. In addition, medical personnel may prescribe medication based solely on the medical personnel's knowledge of classes of drugs without any regard to what drugs are on a particular insurance formulary. Also, medical personnel may determine and provide treatment options based on one or more guidelines that are provided by a governing body and are known to the medical personnel. Such guidelines are based on data points from many past cases of a similar nature and are aimed at providing the best possible treatment to a subject without increasing the subject's risk of contracting a subsequent illness or injury that would require long term care (e.g., a stroke). However, it may be difficult for medical personnel to keep abreast on all guidelines, which are constantly changing.

Accordingly, a need exists for improved approaches to assist medical personnel in making decisions that will result in effective treatment of a subject, while also reducing potential long term care costs.

### SUMMARY

In one embodiment, a system includes one or more data capture devices for collecting captured data that indicates a medical condition of a subject. The system also includes one or more processors and one or more non-transitory memory modules communicatively coupled to the one or more processors and the one or more data capture devices. The memory modules store machine-readable instructions that, when executed, cause the one or more processors to receive the captured data and the image data from the one or more data capture devices. The processors are further caused to complete one or more decision-making processes by analyzing the captured data and the image data. The computing device is trained to analyze the captured data indicating the medical condition of the subject and the visual data pertaining to the at least one characteristic of the subject. The processors are further caused to generate at least one recommendation based on the one or more decision-making processes.

In another embodiment, a method for providing medical recommendations includes receiving, by a computing device, captured data indicating a medical condition of a subject and image data representing visual data pertaining to at least one characteristic of the subject from one or more data capture devices. The method also includes completing, by the computing device, one or more decision-making processes by analyzing the captured data and the image data. The computing device is trained to analyze the captured data indicating the medical condition of the subject and the visual data pertaining to the at least one characteristic of the subject. Finally, the method includes generating at least one recommendation based on the one or more decision-making processes.

In yet another embodiment, a non-transitory, computer-readable storage medium that is operable by a computer for providing medical recommendations is disclosed. The non-transitory, computer-readable storage medium includes one or more programming instructions stored thereon for causing a processing device to receive the captured data and the image data from the one or more data capture devices. The processors are further caused to complete one or more decision-making processes by analyzing the captured data and the image data. The computing device is trained to analyze the captured data indicating the medical condition of the subject and the visual data pertaining to the at least one characteristic of the subject. The processors are further caused to generate at least one recommendation based on the one or more decision-making processes.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a block diagram of the various components of an illustrative computing system for providing suggestions for making medical decisions according to one or more embodiments shown and described herein;

FIG. 2A schematically depicts a block diagram of illustrative computer processing hardware components according to one or more embodiments shown and described herein;

FIG. 2B schematically depicts a block diagram of illustrative logic modules contained within a memory of a computing device according to one or more embodiments shown and described herein;

FIG. 2C schematically depicts a block diagram of other illustrative logic modules contained within a memory of a computing device according to one or more embodiments shown and described herein;

FIG. 2D schematically depicts a block diagram of illustrative data components contained within a data storage device according to one or more embodiments shown and described herein; and

FIG. 3 schematically depicts a flow diagram of an illustrative method of providing suggestions for making medical decisions according to one or more embodiments shown and described herein.

### DETAILED DESCRIPTION

The embodiments described herein are generally directed to systems and methods that obtain data regarding a subject, analyze the data to determine one or more medical issues, and provide one or more suggestions to medical personnel. The data obtained regarding the subject may be medical data, health data, insurance data, and/or the like. The suggestions may be a particular medication that is on a particular formulary of an insurance policy held by the subject, a suggested diagnosis, a suggested treatment plan, a suggested long term care plan, an update to medical guidelines, and/or the like. It should be understood that the present disclosure is not related to a particular field of medicine or medical discipline.

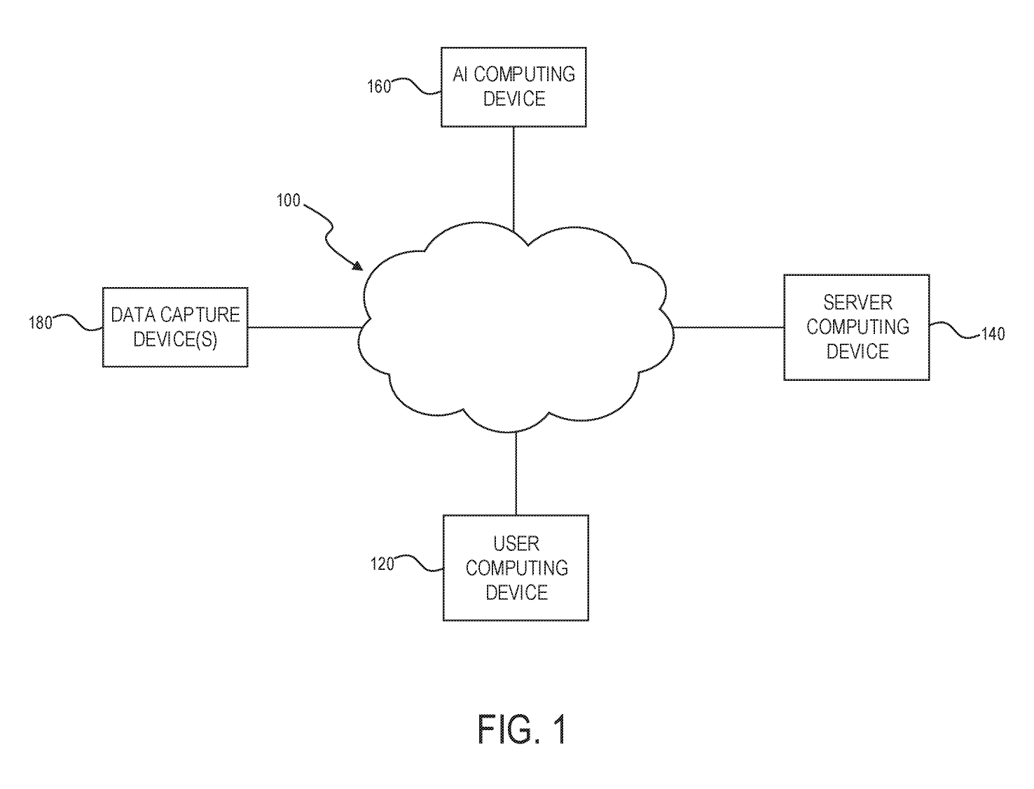

FIG. 1 depicts an illustrative computing network for a system for providing suggestions for making medical decisions according to embodiments shown and described herein. As illustrated in FIG. 1, a computer network 100 may include a wide area network (WAN), such as the Internet, a local area network (LAN), a mobile communications network, a public service telephone network (PSTN), a personal area network (PAN), a metropolitan area network (MAN), a virtual private network (VPN), and/or another network. The computer network 100 may generally be configured to electronically connect one or more computing devices and/or components thereof. Illustrative computing devices may include, but are not limited to, a user computing device 120, a server computing device 140, and an artificial intelligence (AI) computing device 160. In some embodiments, the computer network 100 may also include one or more data capture devices 180, as described in greater detail herein. The user computing device 120 and the data capture device 180 may be separate devices as shown in FIG. 1, or may be integrated into a single device (i.e., the data capture device 180 may be a component of the user computing device 120).

The user computing device 120 may be an interface between a user and the other components connected to the computer network 100, and/or various other components communicatively coupled to the user computing device 120 (such as components communicatively coupled via one or more networks to the user computing device 120, the one or more data capture devices 180, and/or the like), whether or not specifically described herein. Thus, the user computing device 120 may be used to perform one or more user-facing functions, such as receiving inputs from a user or providing information to the user. Additionally, in the event that the server computing device 140 requires updating, correction, and/or the like, the user computing device 120 may provide the desired updating, correction, and/or the like.

The server computing device 140 may receive electronic data and/or the like from one or more sources (e.g., the user computing device 120, the one or more data capture devices 180 and/or one or more databases). In addition, the server computing device 140 may direct operation of one or more other devices (e.g., the one or more data capture devices 180 and/or the user computing device 120), receive data (e.g., from the one or more data capture devices 180 and/or the user computing device 120), provide treatment suggestions, provide long term care suggestions, provide medication suggestions, generate new medical guidelines, update old medical guidelines, and/or the like.

The AI computing device 160 may be a machine that has been particularly configured and trained to parse and analyze data, determine possible diagnoses, treatment options, long term care options, on-formulary medication prescription medications, and/or the like. Some AI computing devices 160 may be maintained by a third party (i.e., a party that does not maintain the various components of the system 100 described herein). Illustrative examples of systems/platforms that may provide the AI computing devices 160 may include, but are not limited to, IBM Watson® (including Watson Analytics™ and/or Watson Health™) (International Business Machines Corp., Armonk, N.Y.), Microsoft® Azure® Data Lake Analytics (Microsoft Corp., Redmond, Wash.), Google Analytics™ and Google Assistant (Alphabet, Inc., Mountain View, Calif.). Other systems/platforms that may provide the AI computing devices 160 should generally be understood.

It should be understood that while the user computing device 120 may be a personal computer, and the server computing device 140 may be a server, these are non-limiting examples. More specifically, some embodiments may be configured with any type of computing device (e.g., mobile computing device, personal computer, server, etc.) to perform the described functionality. Additionally, while each of these computing devices is illustrated in FIG. 1 as a single piece of hardware, this is also merely an example. More specifically, each of the user computing device 120, the server computing device 140, and/or the AI computing device 160 may represent a plurality of computers, servers, databases, components, and/or the like.

It should also be understood that while the embodiments depicted herein refer to a network of computing devices, the present disclosure is not solely limited to such a network. For example, the various processes described herein may be completed by a single computing device, such as a non-networked computing device or a networked computing device that does not use the network to complete the various processes described herein.

The one or more data capture devices 180 are not limited by this disclosure, and may generally be any device that can capture data that can be used for the purposes of analyzing a subject's medical condition. In some embodiments, the one or more data capture devices 180 may include a camera, such as a smartphone camera that is integrated with the user computing device 120. Such a camera can be used to capture image data that is used to obtain information. The image data represents visual data pertaining to at least one characteristic of a patient. For example, a camera may capture an electrocardiogram (ECG) screen that is used to obtain ECG data. In another example, a camera may be used to capture an image of a rash on a subject's skin, which may be used to obtain symptom data. In another example, a camera may provide a digital data feed whereby images of a target object are continuously captured and transmitted. In some embodiments, the one or more data capture devices may be electronic monitoring devices, such as, for example, a fluid pump, a blood pressure cuff, a pulse oximeter, an x-ray machine, a computed tomography scan machine, and/or the like. Such data capture devices capture specific data regarding a subject and transmit the data for analysis as described herein.

While a single data capture device 180 is depicted herein, the number of data capture devices is not limited by this disclosure and may generally be any number thereof. In a non-limiting example, a plurality of data capture devices 180 may be used to capture various types of data regarding a particular subject, or to capture data of a plurality of subjects at substantially the same time.

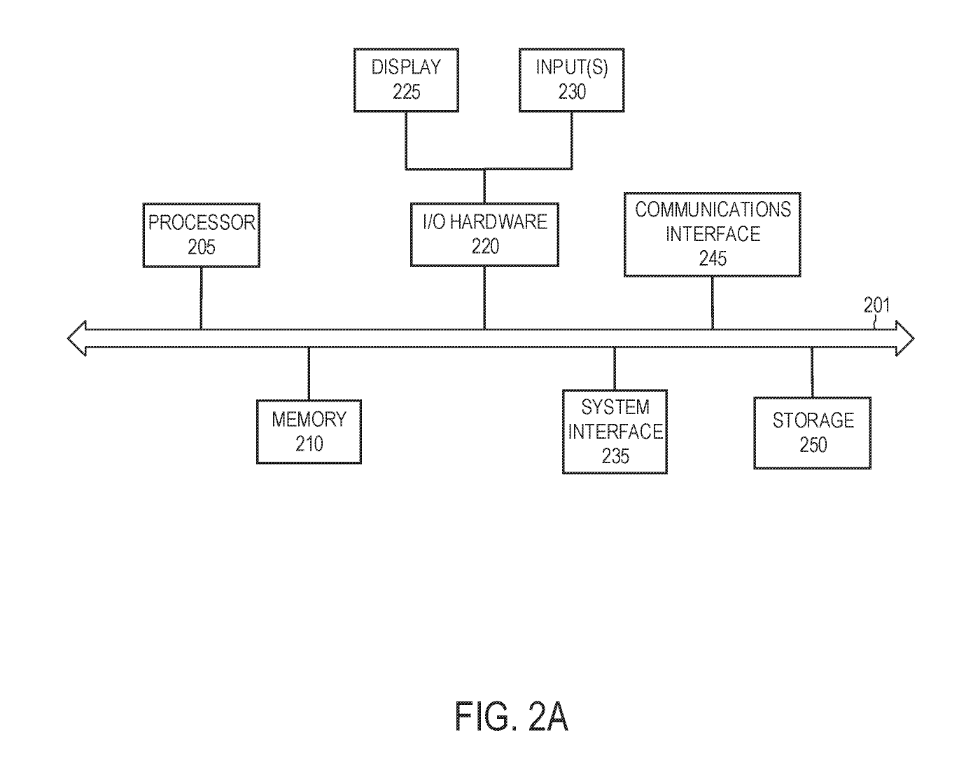

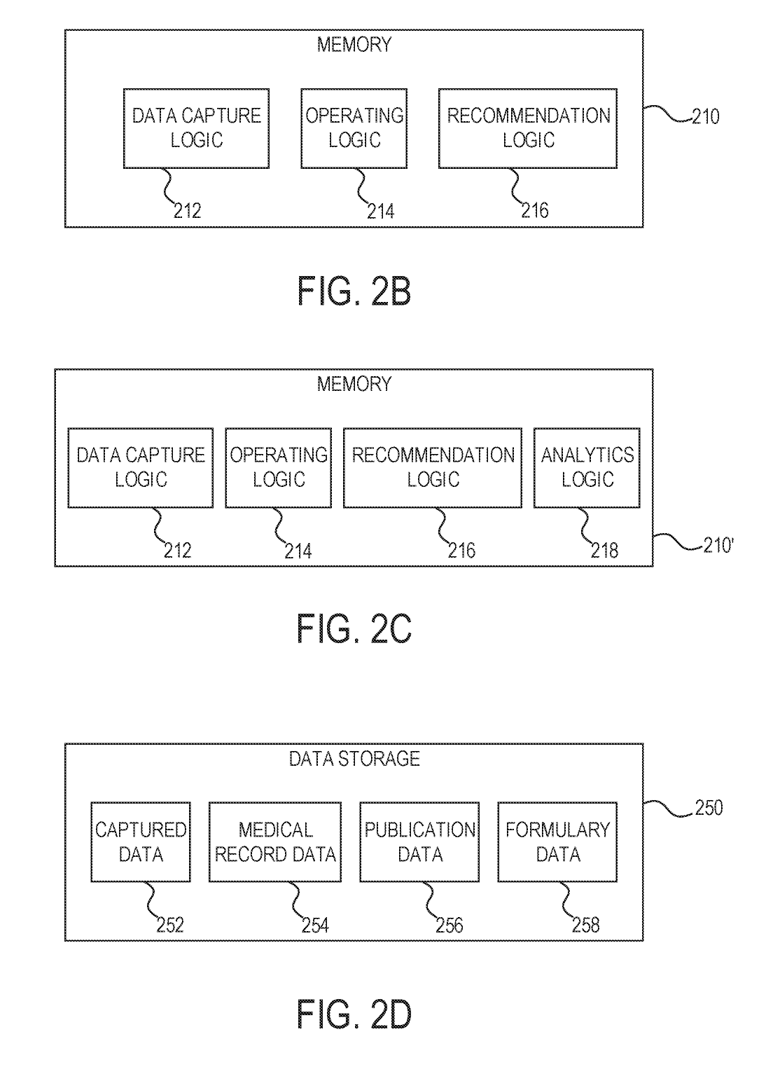

FIG. 2A schematically depicts a block diagram of illustrative hardware of a computing device (e.g., the user computing device 120 of FIG. 1) according to one or more embodiments shown and described herein. While the components described herein are specifically directed to the user computing device 120 of FIG. 1, it should be understood that such components may also be present in the server computing device 140 without departing from the scope of the present disclosure. In some embodiments, a first portion of the components described with respect to FIG. 2A may be contained within the server computing device 140 and a second portion of the components may be contained within the user computing device 120.

A bus 201 may interconnect the various components. A processing device 205, such as a computer processing unit (CPU), may be the central processing unit of the computing device, performing calculations and logic operations to execute a program. The processing device 205, alone or in conjunction with the other components, is an illustrative processing device, computing device, processor, or combination thereof. Memory 210, such as read only memory (ROM) and/or random access memory (RAM), may constitute an illustrative memory device and/or a non-transitory processor-readable storage medium. The memory 210 may include one or more programming instructions thereon that, when executed by the processing device 205, cause the processing device 205 to complete various processes, such as the processes described herein. In some embodiments, the program instructions may be stored on a tangible computer-readable medium that may be removable, such as a compact disc, a digital disk, flash memory, a memory card, a USB drive, an optical disc storage medium, such as a Blu-ray™ disc, and/or other non-transitory processor-readable storage media. Similarly, the program instructions stored on the memory 210 may be embodied as a plurality of software logic modules, where each logic module provides programming instructions for completing one or more tasks, as described in greater detail hereinbelow with respect to FIG. 2B.

A storage device 250, which may generally be a storage medium, may contain one or more data repositories for storing data that is used for storing data that is received (regardless of where the data is received from). The storage device 250 may be any physical storage medium, including, but not limited to, a hard disk drive (HDD), memory, removable storage, and/or the like. While the storage device 250 is depicted as a local device, it should be understood that the storage device 250 may be a remote storage device, such as, for example, a server computing device or the like (e.g., the server computing device 140 of FIG. 1). Illustrative data that may be contained within the storage device 250 is described hereinbelow with respect to FIG. 2D.

Still referring to FIG. 2A, I/O hardware 220 may communicate information from the bus 201 to be displayed on a display 225 portion of the computing device in audio, visual, graphic, or alphanumeric format. Moreover, the I/O hardware 220 may also include one or more inputs 230 that allow for transmission to and receipt of data from input devices such as a keyboard, a mouse, a joystick, a touch screen, a remote control, a pointing device, a video input device, an audio input device, a haptic feedback device, and/or the like. Such an input/output (I/O) hardware 220 may be used, for example, to allow a user to interact with the computing device or any component thereof.

A system interface 235 may generally cause the computing device to interface with one or more of the components of the computer network 100 (FIG. 1), such as, for example, the one or more data capture devices 180 (FIG. 1). Communication with such components may occur using various communication ports (not shown). An illustrative communication port may be attached to a communications network, such as the Internet, an intranet, a local network, a direct connection, and/or the like.

A communications interface 245 may generally cause the computing device to interface with one or more external components, such as, for example, an external computing device, a remote server, and/or the like. Communication with external devices may occur using various communication ports (not shown). An illustrative communication port may be attached to a communications network, such as the Internet, an intranet, a local network, a direct connection, and/or the like.

In some embodiments, the program instructions contained on the memory 210 may be embodied as a plurality of software modules, where each module provides programming instructions for completing one or more tasks. For example, FIG. 2B schematically depicts a block diagram of software modules contained within a memory (e.g., the memory 210) of a computing device according to one or more embodiments shown and described herein. The memory 210 may contain data capture logic 212, operating logic 214, and/or recommendation logic 216. The data capture logic 212 may include one or more software modules for obtaining data regarding a subject, providing data regarding a subject, capturing data regarding a subject, and/or directing operation of the one or more data capture devices 180 (FIG. 1). The operating logic 214 may include an operating system and/or other software for managing components of a computing device. The recommendation logic 216 may include one or more software modules for providing information and/or recommendations to medical personnel for the purposes of making a medical decision, as described in greater detail herein.

Alternatively, as shown in FIG. 2C, memory 210′ contained within other computing devices, such as, for example, the AI computing device 160 (FIG. 1) may also contain analytics logic 218 in addition to the data capture logic 212, the operating logic 214, and/or the recommendation logic 216. The analytics logic 218 may include one or more software modules for analyzing data and making various determinations based on the data, as described in greater detail herein.

FIG. 2D schematically depicts a block diagram of various data contained within a storage device (e.g., the storage device 250) of a computing device according to one or more embodiments shown and described herein. As shown in FIG. 2D, the storage device 250 may include, for example, captured data 252, medical record data 254, publication data 256, and/or formulary data 258. Captured data 252 may include, for example, data that is captured by the one or more data capture devices 180 (FIG. 1) and is subsequently used for evaluation. In some embodiments, the captured data 252 may include data that is received by the one or more data capture devices 180 (FIG. 1) when it is activated to capture data (e.g., a camera is activated to capture an image of an ECG). Medical record data 254 may include, for example, medical data relating to one or more subjects. That is, the medical record data 254 includes data that has been previously stored as a portion of a subject's medical records (e.g., electronic medical record data). Examples of information contained within the medical record data 254 includes, but is not limited to, previous health history, previous surgical procedures, historical laboratory results, diagnosed conditions, current and past medications taken, family history, and/or the like. Publication data 256 may include, for example, data that is contained within publications such as medical journals, data from guidelines, and/or the like. Formulary data 258 includes data regarding certain drugs and medications that are currently on a particular insurance company's formulary (including data regarding drugs and medications that are not currently on a particular insurance company's formulary). Such data may be updated in real-time as new information is received to ensure that the recommendations provided to a medical service provider as described herein reflects the most accurate and up-to-date information.

It should be understood that the components illustrated in FIGS. 2A-2D are merely illustrative and are not intended to limit the scope of this disclosure. More specifically, while the components in FIGS. 2A-2D are illustrated as residing within the user computing device 120, the server computing device 140, and/or the artificial intelligence computing device 160 (FIG. 1), these are non-limiting examples. In some embodiments, one or more of the components may reside external to the user computing device 120, the server computing device 140, and/or the artificial intelligence computing device 160 (FIG. 1). One or more of the components may also be embodied in other computing devices not specifically described herein.

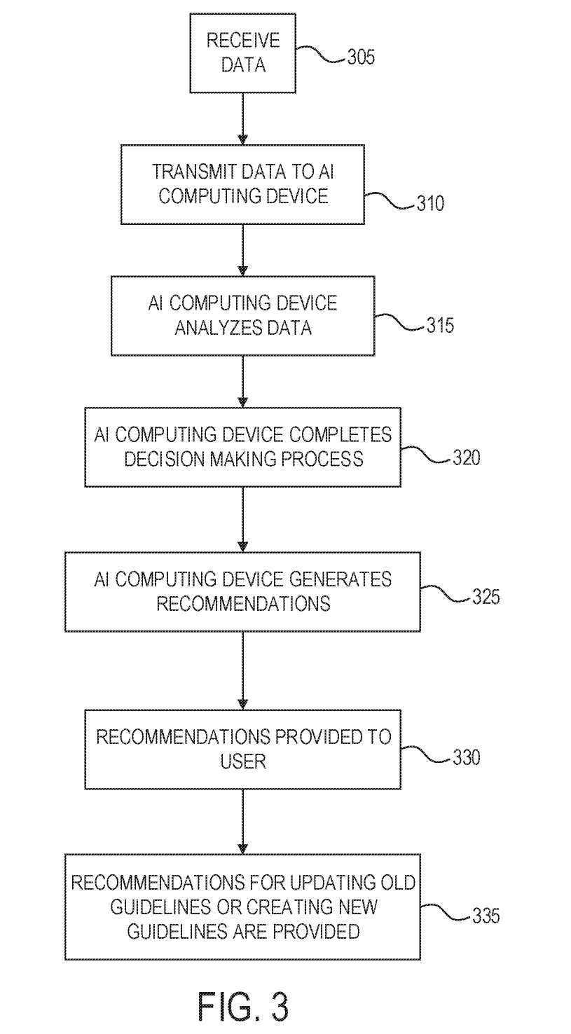

FIG. 3 illustrates an exemplary flow diagram of a method for providing suggestions for making medical decisions. Referring generally to FIGS. 1 and 3, at step 305, the systems 100 described herein receives data regarding a subject. The data may be received from one or more sources, and includes electronic medical record data, biometric sensor data, data entered by the subject, data captured by an imaging device, and/or the like. Electronic medical record data includes a subject's full medical history, prior and current treatments, prior and current medications, current health insurance provider, and/or the like. Biometric sensor data includes data received from any sensors that can obtain information from a subject, such as electrocardiogram (EKG) data, and/or the like. Data entered by the subject may include data entered through a user interface, such as self-reporting data (dates/times of medication intake, food consumption, exercise, etc.). Data may also be obtained by searching available repositories for relevant data. In some embodiments, medical personnel, such as a nurse practitioner, may obtain an image or other data from a biometric device such as an electrocardiograph (ECG) and transmit the data for the purposes of assisting with diagnosis (e.g., for diagnosing atrial fibrillation (AFIB)). In some embodiments, the data may be received via one or more of the data capture devices described herein. In some embodiments, the data may be collected from one of the data repositories described herein.

At step 310, the collected data may be transmitted to the AI computing device 160. At step 315, the data may be analyzed by the AI computing device 160. Additionally, the AI computing device 160 may also complete one or more decision-making processes at step 320. Illustrative examples of decision-making processes may include, but are not limited to, determining current or future health risks for the subject, assisting in confirming diagnoses, recommending medication classes, and/or the like. These tasks may be completed as often as necessary. For example, the processes may be continuously completed as new data regarding the subject is received. In another example, the AI computing device 160 may analyze the medical data, calculate a risk of a potential complication for each potential treatment program, access a guidelines database to determine whether any guidelines exists, search for clinical trials, and provide one or more treatment programs. In addition, the AI computing device 160 may query one or more additional sources of additional data, literature, or the like to obtain additional information that may be used for providing an accurate recommendation. The AI computing device 160 may be particularly trained to determine health risks and potential treatment programs based on the guidelines that have been established by a governing body (e.g., a professional organization) for the purposes of treating a particular illness or injury and/or additional discovered data, while avoiding potential side effects or future illnesses (e.g., stroke). The AI computing device 160 may also provide assistance in diagnosing certain conditions (e.g., AFIB) by confirming possible diagnoses and/or by suggesting potential diagnoses. It should be appreciated that the AI computing device 160 is particularly trained, and therefore is capable of analyzing significantly more data to arrive at a recommendation in a shorter period of time than when compared to conventional techniques that are presently available. As a result, the systems and methods described herein improve the functionality of the various computing devices described herein by allowing them to analyze data more quickly and to arrive at a recommendation in a shorter period of time.

At step 325, the AI computing device 160 may generate recommendations. Recommendations can include the decisions described above and/or recommended medication classes to prescribe recommended potential surgical procedures, recommended therapy aids, recommended monitoring devices, recommended clinical trials, and/or the like. Recommended medication classes may be general classes or specific classes, and may also account for particular insurance formularies and/or practitioners so as to provide the most economical medication recommendations. Recommended clinical trials may be clinical trials or other related programs that are available from registries such as the PINNACLE registry (American College of Cardiology).

The recommendations may be provided to a user at step 330. More specifically, in one embodiment the user may be one or more treatment personnel such as, for example, nurse practitioners, doctors, and/or the like. The recommendations are generally consistent with the guidelines and provide a map framework to treatment personnel for carrying out treatment that is consistent with the guidelines. In one embodiment, the recommendations may be continuously updated and provided to treatment personnel as additional data is received regarding the patient, new guidelines are promulgated, and/or the like.

At step 335, recommendations for updating old guidelines or creating new guidelines based on information that is analyzed may be provided. More specifically, if a guideline predates new data, then a recommendation may be generated for updating the guideline. It should be appreciated that step 335 is optional, and may be omitted in some embodiments.

Referring generally to the figures, the disclosed systems and methods calculate medical recommendations in a non-conventional manner by based on data collected by one or more data capture devices. More specifically, the AI computing device 160 of the system is particularly trained to analyze the data collected by the data capture devices. As mentioned above, the data capture devices may be, but are not limited to, cameras and electronic monitoring devices (e.g., fluid pump, a blood pressure cuff, a pulse oximeter, and the like). Conventional approaches to diagnose a subject may be based solely on the classes of drugs and/or guidelines provided by a governing body that are known by a medical professional. However, sometimes it is difficult to quickly provide an accurate diagnosis based on the limited information known by medical personnel since the guidelines and drug information are in a constant state of flux. In contrast, the disclosed system provides a technical solution to address the issue of constantly changing data by providing a computing system that is trained to parse and analyze data, determine possible diagnoses, treatment options, long term care options, on-formulary medication prescription medications, and/or the like based on artificial intelligence techniques.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A system for providing medical recommendations, the system comprising:
one or more data capture devices configured for collecting captured data and image data, wherein the captured data indicates a medical condition of a subject and the image data represents visual data pertaining to at least one characteristic of the subject;
one or more processors; and
one or more non-transitory memory modules communicatively coupled to the one or more processors and the one or more data capture devices and storing machine-readable instructions that, when executed, cause the one or more processors to perform at least the following:
receive the captured data and the image data from the one or more data capture devices;
complete one or more decision-making processes by analyzing the captured data and the image data, wherein the one or more processors are trained to analyze the captured data indicating the medical condition of the subject and the visual data pertaining to the at least one characteristic of the subject; and
generate at least one recommendation based on the one or more decision-making processes.

2. The system of claim 1, wherein the one or more processors further comprise:
an artificial intelligence computing device trained to determine the at least one recommendation.

3. The system of claim 1, wherein the one or more data capture devices comprise:
a camera configured to capture the image data, the image data being a digital data feed wherein images of a target object are continuously captured and transmitted.

4. The system of claim 1, wherein the one or more data capture devices comprise an electronic monitoring device.

5. The system of claim 4, wherein the electronic monitoring device is one of a fluid pump, a blood pressure cuff, a pulse oximeter, an x-ray machine, and a computed tomography scan machine.

6. The system of claim 1, wherein the medical condition of the subject includes biometric sensor data.

7. The system of claim 1, wherein the one or more non-transitory memory modules store at least one of the captured data, the image data, medical record data, publication data, and formulary data.

8. The system of claim 7, wherein the at least one recommendation is determined based on at least one of the captured data, the medical record data, the publication data, and the formulary data.

9. The system of claim 7, wherein the medical record data comprises at least one of following characteristics of the subject: previous health history, previous surgical procedures, historical laboratory results, diagnosed conditions, current and past medications taken by the subject, and family history.

10. The system of claim 7, wherein the formulary data comprises data regarding drugs and medications that are currently on a formulary for a particular insurance company.

11. The system of claim 1, wherein the at least one recommendation comprises one of the following: future health risks for the subject, assistance in confirming diagnoses, and recommending medication classes.

12. A method for providing medical recommendations, the method comprising:
receiving, by a computing device, captured data indicating a medical condition of a subject and image data representing visual data pertaining to at least one characteristic of the subject from one or more data capture devices;
completing, by the computing device, one or more decision-making processes by analyzing the captured data and the image data, wherein the computing device is trained to analyze the captured data indicating the medical condition of the subject and the visual data pertaining to the at least one characteristic of the subject; and
generating at least one recommendation based on the one or more decision-making processes.

13. The method of claim 12, wherein the method further comprises:
providing an artificial intelligence computing device trained to determine the at least one recommendation.

14. The method of claim 12, further comprising:
Capturing the image data by a camera, the image data being a digital data feed wherein images of a target object are continuously captured and transmitted.

15. The method of claim 12, wherein the one or more data capture devices comprise an electronic monitoring device.

16. The method of claim 15, wherein the electronic monitoring device is one of a fluid pump, a blood pressure cuff, a pulse oximeter, an x-ray machine, and a computed tomography scan machine.

17. The method of claim 12, further comprising:
storing at least one of the captured data, the image data, medical record data, publication data, and formulary data on one or more non-transitory memory modules of the computing device.

18. The method of claim 17, further comprising:
determining the at least one recommendation is determined based on at least one of the captured data, the medical record data, the publication data, and the formulary data.

19. The method of claim 12, wherein the at least one recommendation comprises one of the following: future health risks for the subject, assistance in confirming diagnoses, and recommending medication classes.

20. A non-transitory, computer-readable storage medium that is operable by a computer for providing medical recommendations, the non-transitory, computer-readable storage medium comprising one or more programming instructions stored thereon for causing a processing device to:
receive captured data and image data from one or more data capture devices, wherein the captured data indicates a medical condition of a subject and the image data represents visual data pertaining to at least one characteristic of the subject;
complete one or more decision-making processes by analyzing the captured data and the image data, wherein the computer is trained to analyze the captured data indicating the medical condition of the subject and the visual data pertaining to the at least one characteristic of the subject; and
generate at least one recommendation based on the one or more decision-making processes.
